(19) United States
(12) Patent Application Publication
(10) Pub. No.: US 2020/0158843 A1
(43) Pub. Date: May 21, 2020

(54) METHOD AND SYSTEM FOR OPTIMIZING ULTRASOUND FRAME RATE USING IMPROVED TRANSMIT SEQUENCES

(71) Applicant: GENERAL ELECTRIC COMPANY, Schenectady, NY (US)

(72) Inventors: Heinz Schmied, Zipf (AT); Stephan W. Anzengruber, Zipf (AT); Stefan Denk, Zipf (AT)

(21) Appl. No.: 16/196,715

(22) Filed: Nov. 20, 2018

(51) Int. Cl. G01S 7/52; G01S 15/89; A61B 8/08

(52) U.S. Cl. CPC G01S 7/52046; G01S 7/52095; A61B 8/483; G01S 7/52088; G01S 15/892

(57) ABSTRACT

An ultrasound transmit shot sequence that optimizes frame rate while minimizing image movement artifacts is provided. A method includes transmitting, by an ultrasound probe, a shot sequence having a plurality of shots defining a total number of shots of the shot sequence. The shot sequence includes a minimum spatial distance of shot positions from any of the plurality of shots in the shot sequence to a subsequent shot in the shot sequence that is at least a quarter of the total number of shots. The shot sequence includes a maximum temporal distance between spatially adjacent shots of the plurality of shots that is constant irrespective of the total number of shots in the shot sequence.

### FIELD

Certain embodiments relate to ultrasound imaging. More specifically, certain embodiments relate to a method and system for improving ultrasound transmit shot sequences to optimize an ultrasound frame rate while minimizing stitching artifacts and image artifacts caused by late echoes and reverberations below an intended ultrasound penetration depth.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

The ultrasound frame rate when acquiring brightness mode (B-mode or 2D mode) images may be limited by the physical sound runtime through the penetrated media, including the runtime of both the transmitted sound as well as the reflected echoes. As illustrated in FIG. 1, conventional ultrasound systems typically generate ultrasound images from consecutively transmitted sound waves (known as shots) emitted at adjacent shot positons in an image field. Referring to FIG. 1, an ultrasound probe may sequentially transmit ultrasound shots from an ultrasound probe transducer array surface. An ultrasound shot sequence may begin at a first outer side of an image field and may extend in succession to a second outer side of the image field. The echoes from sound reflections below the intended penetration depth, called late echoes and reverberations, may cause image artifacts to appear in the ultrasound images.

For example, FIG. 2 illustrates an image field having a shot time visible depth portion up until an intended penetration depth and a dead time below visible depth portion that is below the intended penetration depth. As shown in FIG. 2, objects located within the intended penetration depth may appear correctly in the image field. However, objects located below the intended penetration depth may cause artifacts to appear incorrectly in the image field. The artifacts may be due to the ultrasound system interpreting reflections from objects below the intended penetration depth to be reflections from subsequently transmitted ultrasound shots in a shot sequence. In an effort to reduce image artifacts caused by late echoes and reverberations, conventional ultrasound systems may insert a dead time between each ultrasound shot and a subsequent shot in the sequence.

FIG. 3 illustrates an ultrasound shot sequence timing chart corresponding with a portion of the conventional ultrasound shot sequence illustrated in FIG. 1, as is known in the art. Referring to FIG. 3, the timing chart illustrates transmission of a first ultrasound shot, followed by the reception of the echoes corresponding with the first ultrasound shot, and followed by a dead time. After the dead time, the process of transmitting, receiving, and dead time is repeated for second, third, fourth, and fifth ultrasound shots. As shown in FIG. 3, the dead time is typically a fixed value throughout the image sequence in conventional ultrasound systems. The dead time inserted in the ultrasound shot sequence therefore limits the acquisition frame rate of the ultrasound system.

Some ultrasound systems have attempted to overcome the need for dead time by increasing the spatial distance between consecutive shots, such as by alternating the shot sequence as illustrated in FIGS. 4-5. Referring to FIG. 4, a shot sequence alternating between a first side of an image field and a second side of the image field is shown. The shot sequence begins at a first outer side of the image field. The second shot is on an opposite half of the image field adjacent the center of the image field. The third and subsequent odd number shots move from the first shot toward the center. The fourth and subsequent even number shots move from the second shot toward a second outer side, opposite the first outer side, of the image field. Referring to FIG. 5, an ultrasound shot sequence timing chart corresponding with a portion of the ultrasound shot sequence illustrated in FIG. 4 is provided. As shown in FIG. 5, by alternating between right and left halves of the image field, the shot sequence eliminates the need for a dead time because late echoes from a first half of an image field are unlikely to be associated with ultrasound shots from a second half of the image field, and vice versa. However, there is a relatively long time delay due to the temporal distance between acquisition of the two shots at the center of the image field. Specifically, the first shot on the second half of the image field (i.e., the second shot overall) is adjacent the last shot on the first half of the image field (i.e., the eleventh shot overall in the example of FIG. 4). This temporal distance between the acquisition of the two centrally-located adjacent shots often causes stitching artifacts to appear at this central separation line, particularly if there is any movement in the acquisition area. The temporal distance between the acquisition of the two centrally-located adjacent shots increases linearly with the total number of shots in the shot sequence of FIGS. 4-5. For example, the temporal distance from the second shot to the adjacent eleventh shots equals nine (9) when there are twelve (12) total shots as shown in FIG. 4. The temporal distance between the acquisition of the two centrally-located adjacent shots equals twenty-one (21), however, when there are twenty-four (24) total shots (i.e., the temporal distance from the second shot to twenty-third shot in the sequence equals twenty-one (21)).

A solution for eliminating stitching artifacts at the center of the image field by reducing the temporal distance between spatially adjacent shots while still increasing the frame rate is known in the art and illustrated in FIGS. 6-7. Referring to FIG. 6, a shot sequence beginning at a center of an image field and alternating between a first side of the image field and a second side of the image field is shown. The first shot of the shot sequence begins on a first side of the image field adjacent to the center of the image field and each subsequent odd shot moves from the first shot toward a first outer side of the image field. The second shot is on a second side of the image field adjacent to the center of the image field and each subsequent even shot moves from the second shot toward a second outer side, opposite the first outer side, of the image field. Referring to FIG. 7, an ultrasound shot sequence timing chart corresponding with a portion of the ultrasound shot sequence illustrated in FIG. 6 is provided. As shown in FIG. 7, a dead time is inserted for at least the first several shots because these initial shots are spatially close to each other. However, as the spatial separation between the shots increases as the shots move toward the outer sides of the image field, the amount of dead time can be reduced and eventually eliminated. The shot sequence of FIGS. 6-7 may achieve frame rates higher than the conventional shot sequence of FIGS. 1 and 3, and is more effective than the shot sequence of FIGS. 4-5 at preventing stitching artifacts by maintaining a constant maximum temporal distance of two (2) irrespective of a total number of shots. The shot sequence of FIGS. 6-7 does not, however, achieve frame rates as high as the shot sequence of FIGS. 4-5 because of the dead time inserted after early shots in the shot sequence.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for improving ultrasound transmit shot sequences to optimize an ultrasound frame rate while minimizing image movement artifacts, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 illustrates an ultrasound shot sequence beginning at a first outer side of an image field and sequentially advancing to a second outer side of the image field, as known in the art.

FIG. 2 illustrates exemplary appearances of various objects in an image field based on the ultrasound shot sequence illustrated in FIG. 1, as known in the art.

FIG. 3 illustrates an ultrasound shot sequence timing chart corresponding with a portion of the ultrasound shot sequence illustrated in FIG. 1, as known in the art.

FIG. 4 illustrates an ultrasound shot sequence alternating between a first side of an image field and a second side of the image field, the odd number shots on the first side beginning at a first outer side of the image field and advancing toward the center of the image field, and the even number shots on the second side beginning at the center of the image field and advancing toward a second outer side of the image field, as known in the art.

FIG. 5 illustrates an ultrasound shot sequence timing chart corresponding with a portion of the ultrasound shot sequence illustrated in FIG. 4, as known in the art.

FIG. 6 illustrates an ultrasound shot sequence alternating between a first side of an image field and a second side of the image field, the odd number shots on the first side beginning at a center of the image field and advancing toward a first outer side of the image field, and the even number shots on the second side beginning at the center of the image field and advancing toward a second outer side of the image field, as known in the art.

FIG. 7 illustrates an ultrasound shot sequence timing chart corresponding with a portion of the ultrasound shot sequence illustrated in FIG. 6, as known in the art.

FIG. 8 is a block diagram of an exemplary ultrasound system that is operable to provide an improved ultrasound transmit shot sequence to optimize an ultrasound frame rate while minimizing image movement artifacts, in accordance with various embodiments.

FIG. 9 illustrates an exemplary improved ultrasound shot sequence configured to optimize an ultrasound frame rate while minimizing image movement artifacts, in accordance with various embodiments.

FIG. 10 illustrates an ultrasound shot sequence timing chart corresponding with a portion of the exemplary improved ultrasound shot sequence illustrated in FIG. 9, in accordance with various embodiments.

FIG. 11 illustrates a chart of an exemplary improved ultrasound shot sequence configured to optimize an ultrasound frame rate while minimizing image movement artifacts, in accordance with various embodiments.

FIG. 12 is a flow chart illustrating exemplary steps for execution of a first portion of an exemplary improved ultrasound shot sequence configured to optimize an ultrasound frame rate while minimizing image movement artifacts, in accordance with various embodiments.

FIG. 13 is a flow chart illustrating exemplary steps for execution of a second portion of an exemplary improved ultrasound shot sequence configured to optimize an ultrasound frame rate while minimizing image movement artifacts, in accordance with various embodiments.

### DETAILED DESCRIPTION

Certain embodiments may be found in a method and system for providing an improved ultrasound transmit shot sequence to optimize an ultrasound frame rate while minimizing late echo and stitching image artifacts. Various embodiments have the technical effect of simultaneously increasing ultrasound B-mode image acquisition frame rate while avoiding both late echo artifacts and stitching artifacts by implementing a shot sequence having both a minimum spatial distance from any shot in the sequence to the subsequent shot in the sequence that is at least a quarter of the total number of shots and a constant maximum temporal distance between spatially adjacent shot positions irrespective of a total number of shots in the shot sequence. The minimum spatial distance prevents late echo artifacts thereby allowing the reduction and/or elimination of dead times to increase frame rate while the constant maximum temporal distance irrespective of the total number of shots prevents stitching artifacts.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD, CW where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

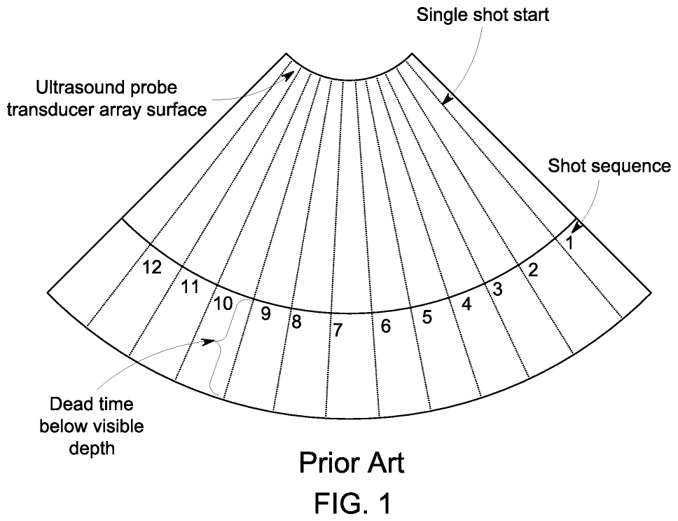

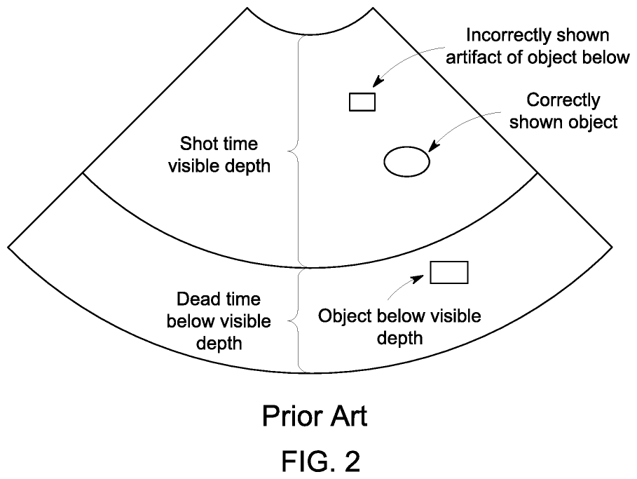

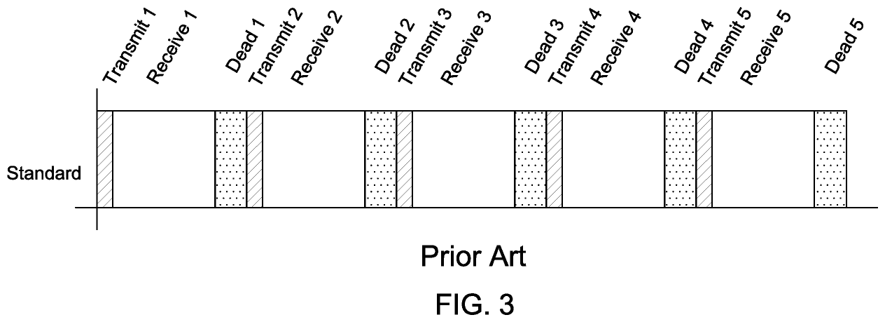

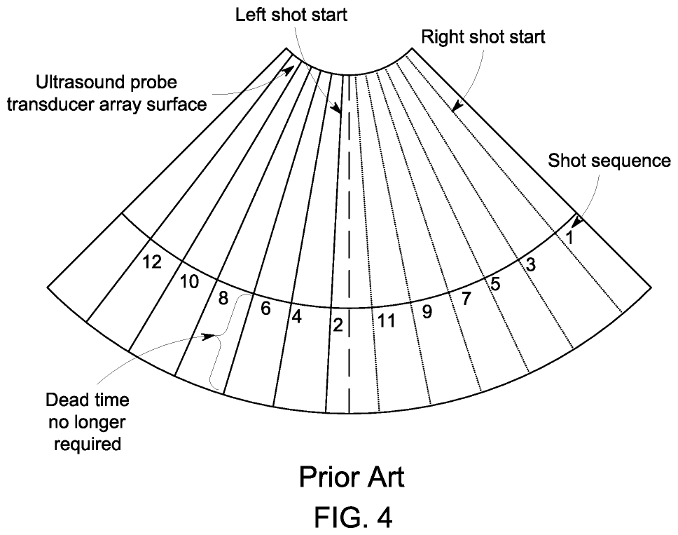

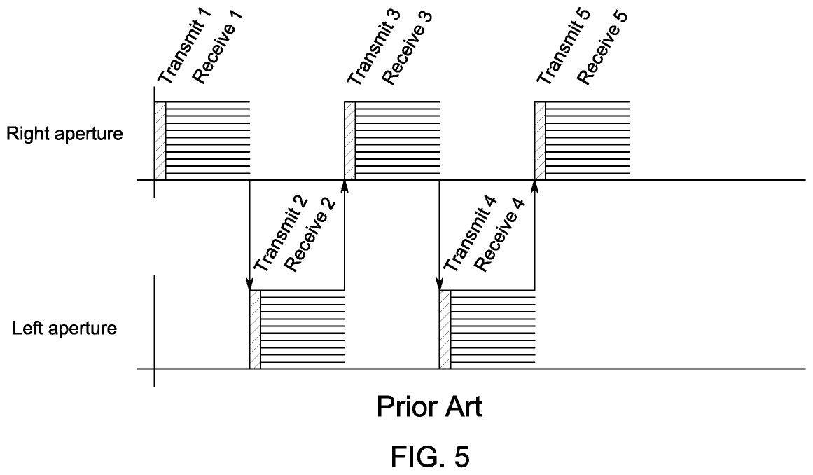

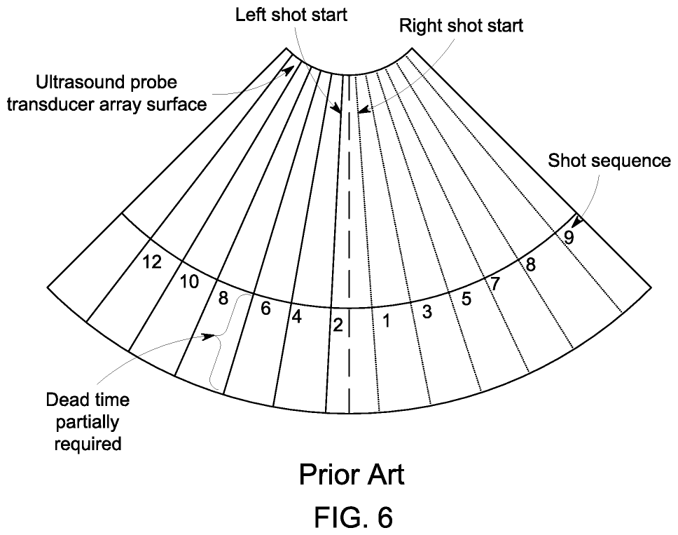

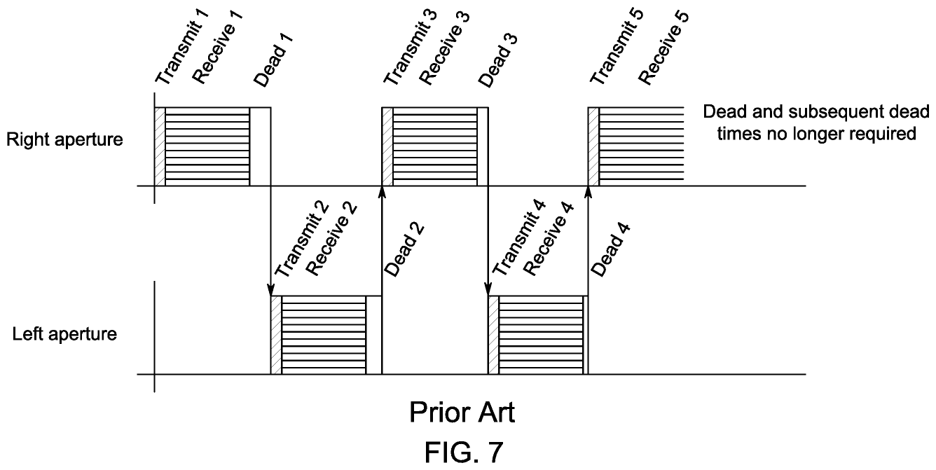

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

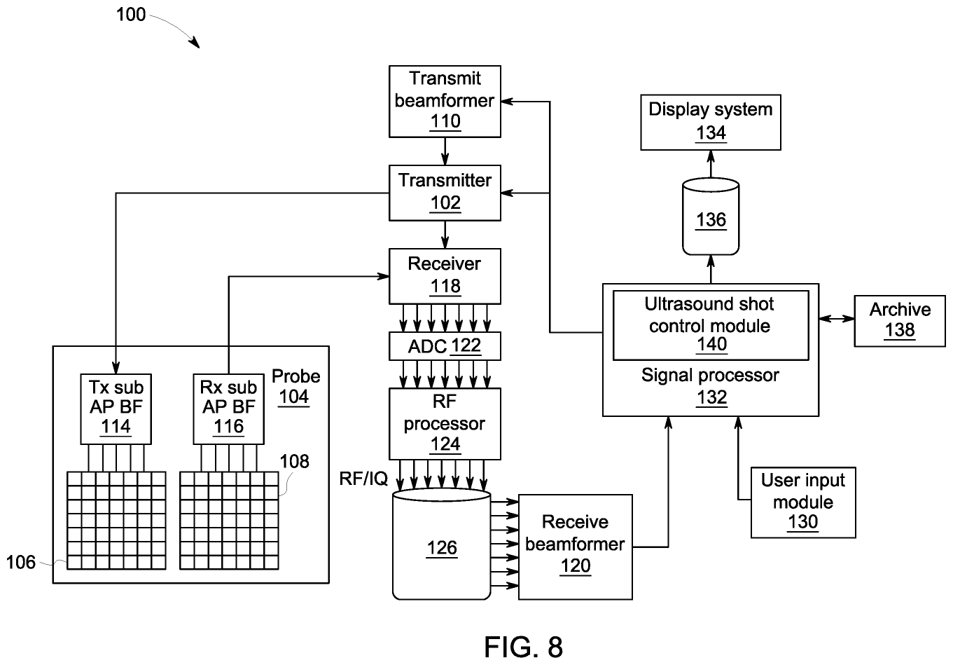

FIG. 8 is a block diagram of an exemplary ultrasound system 100 that is operable to provide an improved ultrasound transmit shot sequence 220, 400 to optimize an ultrasound frame rate while minimizing image movement artifacts, in accordance with various embodiments. Referring to FIG. 8, there is shown an ultrasound system 100. The ultrasound system 100 comprises a transmitter 102, an ultrasound probe 104, a transmit beamformer 110, a receiver 118, a receive beamformer 120, a RF processor 124, a RF/IQ buffer 126, a user input module 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 may comprise a plurality of receive beamformers 120, such as when performing Multi-Gated Doppler (MGD) imaging.

The user input module 130 may be utilized to input patient data, scan parameters, settings, select protocols and/or templates, and the like. In an exemplary embodiment, the user input module 130 may be operable to configure, manage and/or control operation of one or more components and/or modules in the ultrasound system 100. In this regard, the user input module 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, the display system 134, and/or the archive 138. The user input module 130 may include button(s), rotary encoder(s), a touchscreen, motion tracking, voice recognition, a mousing device, keyboard, camera and/or any other device capable of receiving a user directive. In certain embodiments, one or more of the user input modules 130 may be integrated into other components, such as the display system 134, for example. As an example, user input module 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform compounding, motion tracking, and/or speckle tracking. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information. In the exemplary embodiment, the signal processor 132 may comprise an ultrasound shot control module 140.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores medical image data and ultrasound transmit shot sequence instructions, for example.

The ultrasound system 100 may be operable to continuously acquire ultrasound scan data at a frame rate that is suitable for the imaging situation in question. Typical frame rates range from 20-70 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

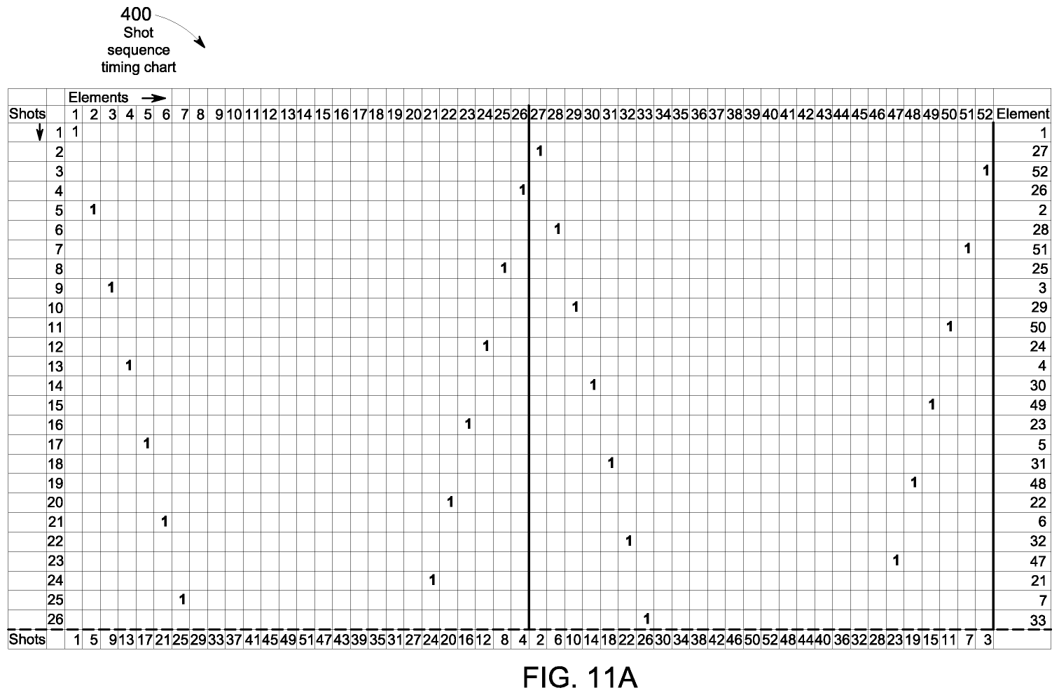

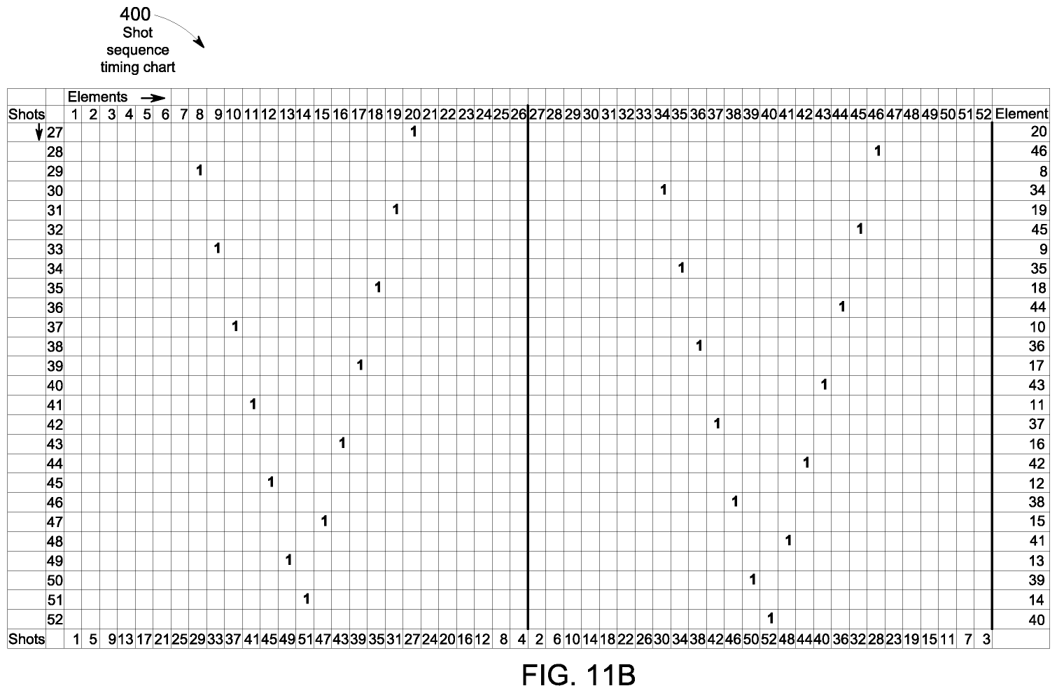

The signal processor 132 may include an ultrasound shot control module 140 that comprises suitable logic, circuitry, interfaces and/or code that may be operable to control and/or otherwise provide instructions to the transmitter 102, transmit beamformer 110, and/or the probe 104 for executing an improved ultrasound transmit shot sequence to optimize an ultrasound frame rate while minimizing image movement artifacts. For example, the shot sequence may distribute ultrasound shots over four zones. The shot sequence may include at least two portions. In embodiments having a first portion and a second portion, for example, each of the portions may be made up of substantially half (defined as 40-60%) of the total shots. Each portion of the shot sequence may include a unique shot pattern. For example, a first portion of the shot sequence may include a first pattern and a second portion of the shot sequence may include a second pattern that is different than the first pattern. As an example, the first pattern may alternate between shots fired in a first zone, third zone, fourth zone, and second zone, which is repeated until the substantially first half of the total shots are fired. The second pattern may alternate between shots fired in the fourth zone, first zone, third zone, and second zone, which is repeated until the substantially second half of the total shots are fired (i.e., the remaining shots of the shot sequence). The second pattern may begin at an appropriate shot in view of the last shot of the first portion. For example, if the last shot in the first portion of the shot sequence was in the third zone, the first shot in the second portion may begin in the second zone because a shot in the second zone follows a shot in the third zone according to the second pattern. The change in the pattern between the first portion and the second portion may ensure a minimum spatial distance from any shot in the sequence to its subsequent shot in the sequence that is at least a quarter of the total number of shots while ensuring a constant maximum temporal distance between spatially adjacent shot positions irrespective of a total number of shots in the shot sequence. For example, as discussed below in connection with FIG. 11, if there are 52 total shots, the minimum spatial distance from any shot in the sequence to its subsequent shot in the sequence would be 13 shot positions and the maximum temporal distance between spatially adjacent shot positions is 5. The minimum spatial distance from any shot in the sequence to its subsequent shot in the sequence that is at least a quarter of the total number of shots may, in many cases depending on the probe type and probe settings, allows a system to eliminate dead time between subsequent shots. The minimum spatial distance from any shot in the sequence to its subsequent shot in the sequence that is at least a quarter of the total number of shots may eliminate late echo image artifacts. The maximum temporal distance of 5 between spatially adjacent shot positions may eliminate stitching image artifacts. In various embodiments, the maximum temporal distance is constant, irrespective of a total number of shots in the shot sequence. In certain embodiments, the constant value of the maximum temporal distance of a shot sequence does not exceed 7.

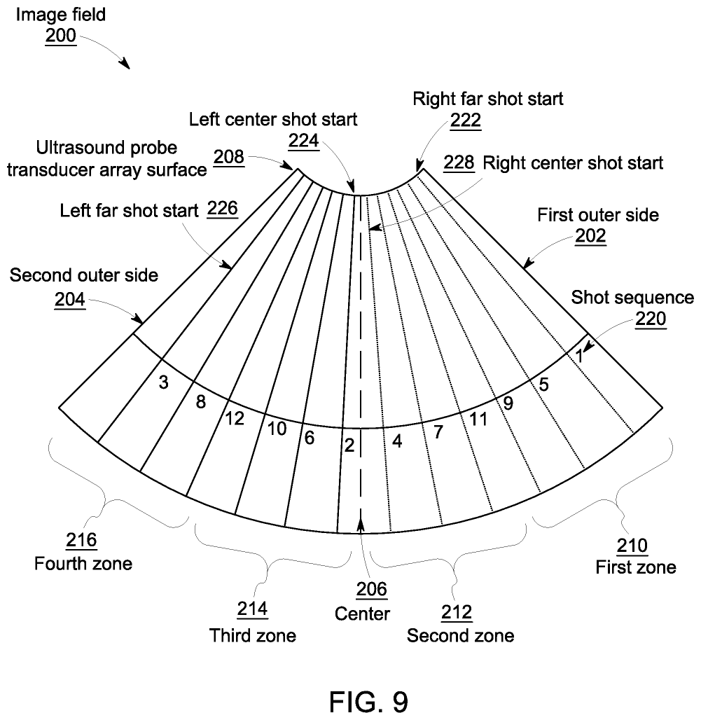

FIG. 9 illustrates an exemplary improved ultrasound shot sequence 220 configured to optimize an ultrasound frame rate while minimizing image movement artifacts, in accordance with various embodiments. Referring to FIG. 9, an image field 200 is provided having an ultrasound probe transducer array surface 208 from which ultrasound shots 222-228 are fired in a shot sequence 220 between a first outer side 202 and a second outer side 204 of the image field 200. The image field 200 comprises four zones 210-216 between the first outer side 202 and the second outer side 204. Two of the zones 210, 212 may be positioned between the first outer side 202 and a center 206 of the image field 200. For example, one of the zones 210 may be positioned adjacent the first outer side 202 and the other zone 212 may be positioned adjacent the center 206. The zones 210, 212 are adjacent to each other and non-overlapping. The other two zones 214, 216 may be positioned between the center 206 and the second outer side 204. For example, one of the zones 216 may be positioned adjacent the second outer side 204 and the other zone 214 may be positioned adjacent the center 206. The zones 214, 216 are adjacent to each other and non-overlapping.

Still referring to FIG. 9, the shot sequence 220 may begin with an ultrasound shot 222 in a first zone 210 adjacent to the first outer side 202 and with each subsequent shot in the first zone 210 moving from the first outer side 202 toward the center 206 of the image field 200. The next shot 224 in the shot sequence 220 may be fired in the third zone 214 on the opposite side of the center 206 of the image field 200 than the first zone 210 and adjacent the center 206. Each of the subsequent shots in the third zone 214 may move from the center 206 of the image field 200 toward the second outer side 204. The following ultrasound shot 226 may be transmitted in the fourth zone 216 adjacent the second outer side 204 of the image field 200 with each of the subsequent shots in the fourth zone 216 moving from the second outer side 204 toward the center 206 of the image field 200. The subsequent shot 228 in the shot sequence 220 may be fired in the second zone 212 adjacent the center 206 of the image field 200 with each subsequent shot in the second zone 212 moving from the center 206 toward the first outer side 202. The shot sequence 220 may then repeat the sequence of first zone 210, third zone 214, fourth zone 216, and second zone 212 for a first substantially half of the transmitted ultrasound shots. Although the shot sequence 220 is shown beginning in the first zone 210, the shot sequence could begin in any of the four zones 210-216 and proceed in the same order as described above.

Once the first substantially half of the total shots have been fired, the shot sequence 220 changes to a second pattern for the remaining shots. For example, after the sixth total shot in the third zone 214 shown in FIG. 9, the seventh shot in the sequence 220 is in the second zone 212, followed by the fourth zone 216, first zone 210, and back to the third zone 214. The second pattern of third zone 214, second zone 212, fourth zone 216, and first zone 210 repeats until all of the shots of the sequence 220 have been fired.

The total number of shots in the exemplary improved ultrasound shot sequence 220 of FIG. 9 is 12 total shots. The minimum spatial distance from any shot in the sequence to its subsequent shot in the sequence is 3 (i.e., the spatial distance from the sixth shot to the seventh shot). The maximum temporal distance between spatially adjacent shot positions is 5 (i.e., the temporal distance from the third shot to the eighth shot). Accordingly, the exemplary improved ultrasound shot sequence 220 of FIG. 9 provides a minimum spatial distance from any shot in the sequence to its subsequent shot in the sequence that is at least a quarter of the total number of shots while ensuring a maximum temporal distance of 5 between spatially adjacent shot positions such that the system may eliminate or reduce dead time while preventing late echo and stitching image artifacts.

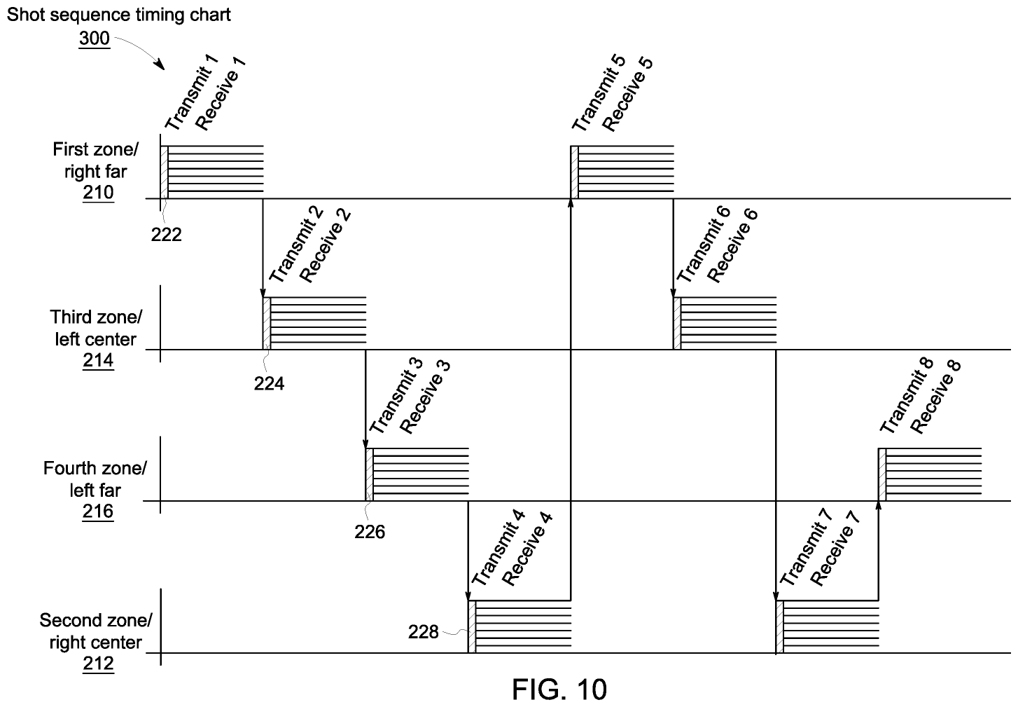

FIG. 10 illustrates an ultrasound shot sequence timing chart 300 corresponding with a portion of the exemplary improved ultrasound shot sequence 220 illustrated in FIG. 9, in accordance with various embodiments. Referring to FIG. 10, after transmission of each ultrasound shot 222-228 is a time period for receiving echoes corresponding to the respective shot. The subsequent shot in the sequence is fired without the insertion of any dead time between the shots. The shot sequence 220 may follow a first pattern for the first six shots prior to assuming a second pattern with the seventh and subsequent shots as discussed above with regard to FIG. 9.

FIG. 11 illustrates a chart of an exemplary improved ultrasound shot sequence 400 configured to optimize an ultrasound frame rate while minimizing image movement artifacts, in accordance with various embodiments. Referring to FIG. 11, the shot sequence 400 includes 52 shots alternating between a first zone, third zone, fourth zone, and second zone for the first 26 shots. After the first substantially half of the shot sequence 400, the shot sequence 400 changes to a pattern of second zone, fourth zone, first zone, and third zone for the last 26 shots in the sequence 400. As illustrated in FIG. 11, the total number of shots in the exemplary improved ultrasound shot sequence 400 is 52 total shots. The minimum spatial distance from any shot in the sequence to its subsequent shot in the sequence is 13 (i.e., the spatial distance from the twenty-sixth shot at element 33 to the twenty-seventh shot at element 20). The maximum temporal distance between spatially adjacent shot positions is 5 (i.e., the temporal distance of the twenty-third shot at element 47 and the twenty-eighth shot at element 46). Accordingly, the exemplary improved ultrasound shot sequence 400 of FIG. 11 provides a minimum spatial distance from any shot in the sequence to its subsequent shot in the sequence that is at least a quarter of the total number of shots while ensuring a maximum temporal distance of 5 between spatially adjacent shot positions such that the system may eliminate or reduce dead time while preventing late echo and stitching image artifacts.

In various embodiments, the maximum temporal distance of a shot sequence 220, 400 is constant irrespective of the number of total shots. For example, FIGS. 9 and 11 illustrate shot sequences 220, 400 having first and second patterns. The first pattern in both FIGS. 9 and 11 are the same (i.e., first zone, third zone, fourth zone, and second zone for a first substantially half of the total shots). The second pattern in both FIGS. 9 and 11 are also the same (i.e., second zone, fourth zone, first zone, and third zone for the last substantially half of the shots). Although the total number of shots in FIG. 9 is 12 and the total number of shots in FIG. 11 is 52, the maximum temporal distance of the shot sequences 220, 400 is constant. Specifically, the maximum temporal distance of the shot sequences 220, 400 of FIGS. 9 and 11 is 5. FIGS. 9 and 11 further illustrate that the minimum spatial distance from any shot in the sequence 220, 400 to its subsequent shot in the sequence 220, 400 is at least a quarter of the total number of shots. Specifically, the total number of shots in FIG. 9 is 12 and the minimum spatial distance is 3. The total number of shots in FIG. 11 is 52 and the minimum spatial distance is 13.

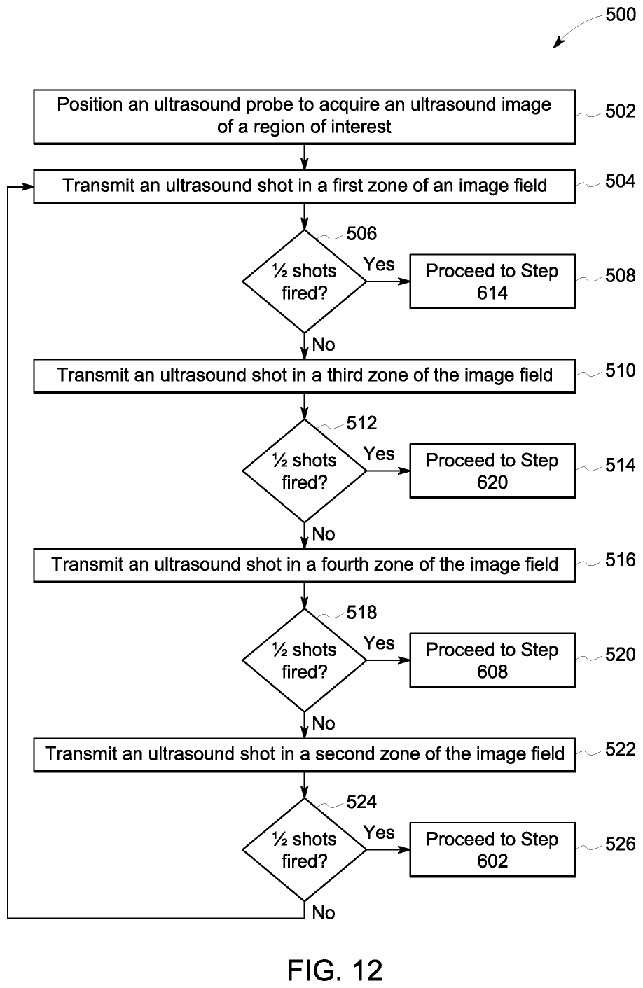

FIG. 12 is a flow chart 500 illustrating exemplary steps 502-526 for execution of a first portion of an exemplary improved ultrasound shot sequence 220, 400 configured to optimize an ultrasound frame rate while minimizing image movement artifacts, in accordance with various embodiments. Referring to FIG. 12, there is shown a flow chart 500 comprising exemplary steps 502 through 526. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, a probe 104 of an ultrasound system 100 may be positioned to acquire a two-dimensional (2D) image of a region of interest. For example, the ultrasound system 100 may acquire the 2D image, such as a B-mode image, with an ultrasound probe 104 positioned over a region of interest, such as a fetus, blood vessel, heart, or any suitable organ or anatomical structure.

At step 504, the probe 104 of the ultrasound system 100 may transmit an ultrasound shot in a first zone 210 of an image field 200. For example, the image field may include four zones 210-216 including a first zone 210, a second zone 212, a third zone 214, and a fourth zone 216. Each of the four zones may include a substantially same amount of ultrasound shot positions. For example, if the image field 200 includes 256 shot positions corresponding to 256 elements of a probe 104, each of the zones 210-216 may include 64 shot positions. The first 210 and second 212 zones may be located between a center 206 of the image field 200 and a first outer side 202 of the image field 200. As an example, one of the first 210 and second 212 zones may be adjacent the first outer side 202 of the image field 200 and the other may be adjacent the center 206 of the image field 200. The third 214 and fourth 216 zones may be located between a center 206 of the image field 200 and a second outer side 204, opposite the first outer side 202, of the image field 200. As an example, one of the third 214 and fourth 216 zones may be adjacent the second outer side 204 of the image field 200 and the other may be adjacent the center 206 of the image field 200. As shown in FIG. 9, the first zone 210 may be adjacent the first outer side 202, the second 212 and third 214 zones may be on opposite sides of the center 206, and the fourth zone 216 may be adjacent the second outer side 204. In various embodiments, ultrasound shots transmitted in the first zone 210 shown in FIG. 9 begin nearest the first outer side 202 with each subsequent shot in the first zone 210 moving from the first outer side 202 toward the center 206 of the image field 200.

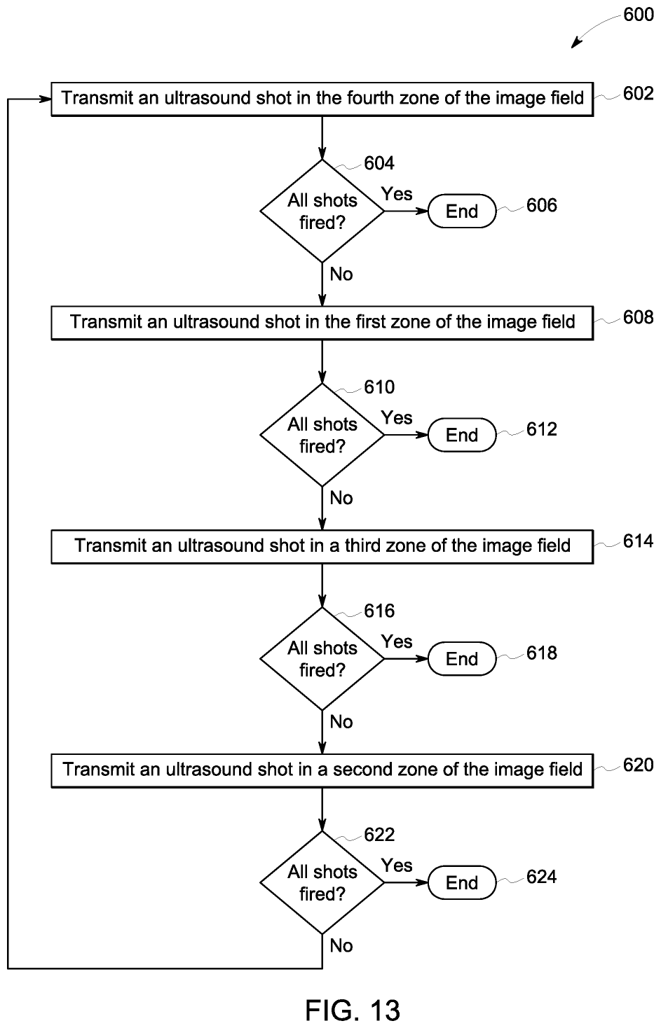

At step 506, the ultrasound system 100 may determine whether substantially half of the shots of the shot sequence 220, 400 have been fired. For example, the shot sequence 220, 400 may have a first portion and a second portion. The shot sequence 220, 400 may be a pre-defined sequence of ultrasound shots. The first portion of the shot sequence 220, 400 may account for substantially half (defined as 40-60%) of the total shots of the shot sequence 220, 400 and the second portion of the shot sequence 220, 400 may account for the other substantially half of the total shots of the shot sequence 220, 400. If the first portion of the shot sequence 220, 400 has been completed after transmitting the ultrasound shot in the first zone 210 of the image field 200 at step 504, ultrasound system 100 at step 506 proceeds to execute the second portion of the shot sequence by proceeding to step 614 of FIG. 13, as defined by step 508. Alternatively, if the first portion of the shot sequence 220, 400 has not been completed after transmitting the ultrasound shot in the first zone 210 of the image field 200 at step 504, ultrasound system 100 at step 506 proceeds to step 510.

At step 510, the probe 104 of the ultrasound system 100 may transmit an ultrasound shot in a third zone 214 of an image field 200. For example, as shown in FIG. 9, the third zone 214 may be adjacent the center 206 of the image field 200 on an opposite side of the image field 200 than the first 210 and second 212 zones. In various embodiments, ultrasound shots transmitted in the third zone 214 shown in FIG. 9 begin nearest the center 206 of the image field 200 with each subsequent shot in the third zone 214 moving from the center 206 toward the second outer side 204, opposite the first outer side 202, of the image field 200. In certain embodiments, the minimum spatial distance from the shot at step 504 to the subsequent shot at step 510 is at least one quarter of the total number of shots in the pre-determined shot sequence 220, 400. For example, if the shot sequence 220, 400 includes a total of 52 shots as illustrated in FIG. 11, the minimum spatial distance from the shot at step 504 to the shot at step 510 is 13 shot positions. In an exemplary embodiment, no dead time is inserted between a reception of echoes from the shot at step 504 and the transmission of the shot at step 510 as illustrated, for example, in FIG. 10.

At step 512, the ultrasound system 100 may determine whether substantially half of the shots of the shot sequence 220, 400 have been fired. For example, similar to step 506, if the first portion of the shot sequence 220, 400 has been completed after transmitting the ultrasound shot in the third zone 214 of the image field 200 at step 510, ultrasound system 100 at step 512 proceeds to execute the second portion of the shot sequence by proceeding to step 620 of FIG. 13, as defined by step 514. Alternatively, if the first portion of the shot sequence 220, 400 has not been completed after transmitting the ultrasound shot in the third zone 214 of the image field 200 at step 510, ultrasound system 100 at step 512 proceeds to step 516.

At step 516, the probe 104 of the ultrasound system 100 may transmit an ultrasound shot in a fourth zone 216 of an image field 200. For example, as shown in FIG. 9, the fourth zone 216 may be adjacent the second outer side 204 of the image field 200 on an opposite side of the image field 200 than the first 210 and second 212 zones. In various embodiments, ultrasound shots transmitted in the fourth zone 216 shown in FIG. 9 begin nearest the second outer side 204 with each subsequent shot in the fourth zone 216 moving from the second outer side 204 toward the center 206 of the image field 200. In certain embodiments, the minimum spatial distance from the shot at step 510 to the subsequent shot at step 516 is at least one quarter of the total number of shots in the pre-determined shot sequence 220, 400. In an exemplary embodiment, no dead time is inserted between a reception of echoes from the shot at step 510 and the transmission of the shot at step 516 as illustrated, for example, in FIG. 10.

At step 518, the ultrasound system 100 may determine whether substantially half of the shots of the shot sequence 220, 400 have been fired. For example, similar to steps 506 and 512, if the first portion of the shot sequence 220, 400 has been completed after transmitting the ultrasound shot in the fourth zone 216 of the image field 200 at step 516, ultrasound system 100 at step 518 proceeds to execute the second portion of the shot sequence by proceeding to step 608 of FIG. 13, as defined by step 520. Alternatively, if the first portion of the shot sequence 220, 400 has not been completed after transmitting the ultrasound shot in the fourth zone 216 of the image field 200 at step 516, ultrasound system 100 at step 518 proceeds to step 522.

At step 522, the probe 104 of the ultrasound system 100 may transmit an ultrasound shot in a second zone 212 of an image field 200. For example, as shown in FIG. 9, the second zone 214 may be adjacent the center 206 of the image field 200 on an opposite side of the image field 200 than the third 214 and fourth 216 zones. In various embodiments, ultrasound shots transmitted in the second zone 212 shown in FIG. 9 begin nearest the center 206 of the image field 200 with each subsequent shot in the second zone 212 moving from the center 206 toward the first outer side 202, opposite the second outer side 204, of the image field 200. In certain embodiments, the minimum spatial distance from the shot at step 516 to the subsequent shot at step 522 is at least one quarter of the total number of shots in the pre-determined shot sequence 220, 400. In an exemplary embodiment, no dead time is inserted between a reception of echoes from the shot at step 516 and the transmission of the shot at step 522 as illustrated, for example, in FIG. 10.

At step 524, the ultrasound system 100 may determine whether substantially half of the shots of the shot sequence 220, 400 have been fired. For example, similar to steps 506, 512, and 518, if the first portion of the shot sequence 220, 400 has been completed after transmitting the ultrasound shot in the second zone 212 of the image field 200 at step 522, ultrasound system 100 at step 524 proceeds to execute the second portion of the shot sequence by proceeding to step 602 of FIG. 13, as defined by step 526. Alternatively, if the first portion of the shot sequence 220, 400 has not been completed after transmitting the ultrasound shot in the second zone 212 of the image field 200 at step 522, ultrasound system 100 at step 524 returns to step 504 to transmit a next ultrasound shot in the first zone 210 of the image field 200. In certain embodiments, the minimum spatial distance from the shot at step 522 to the subsequent shot at step 504 is at least one quarter of the total number of shots in the pre-determined shot sequence 220, 400. In an exemplary embodiment, no dead time is inserted between a reception of echoes from the shot at step 522 and the transmission of the shot at step 504 as illustrated, for example, in FIG. 10. The flow chart 500 continues to repeat steps 504-526 until the first portion of the shot sequence 220, 400 is complete. In various embodiments, the maximum temporal distance between spatially adjacent shot positions is constant, irrespective of the total number of shots in the shot sequence 220, 400. For example, in various embodiments, the maximum temporal distance between spatially adjacent shot positions may be 5. The method then proceeds to the appropriate step in the flow chart 600 of FIG. 13 as described below to carry out the second portion of the shot sequence 220, 400. In certain embodiments, the minimum spatial distance from the last shot in the first portion of the shot sequence 220, 400 to the subsequent shot in the second portion of the shot sequence 220, 400 is at least one quarter of the total number of shots in the pre-determined shot sequence 220, 400. In an exemplary embodiment, no dead time is inserted between a reception of echoes from the last shot in the first portion of the shot sequence 220, 400 and the transmission of the subsequent shot in the second portion of the shot sequence 220, 400.

FIG. 13 is a flow chart 600 illustrating exemplary steps 602-624 for execution of a second portion of an exemplary improved ultrasound shot sequence 220, 400 configured to optimize an ultrasound frame rate while minimizing image movement artifacts, in accordance with various embodiments. Referring to FIG. 13, there is shown a flow chart 600 comprising exemplary steps 602 through 624. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 602, a probe 104 of an ultrasound system 100 may transmit an ultrasound shot in a fourth zone 216 of an image field 200. For example, the image field may include four zones 210-216 including a first zone 210, a second zone 212, a third zone 214, and a fourth zone 216. Each of the four zones may include a substantially same amount of ultrasound shot positions. The first 210 and second 212 zones may be located between a center 206 of the image field 200 and a first outer side 202 of the image field 200. The third 214 and fourth 216 zones may be located between a center 206 of the image field 200 and a second outer side 204, opposite the first outer side 202, of the image field 200. As shown in FIG. 9, the first zone 210 may be adjacent the first outer side 202, the second 212 and third 214 zones may be on opposite sides of the center 206, and the fourth zone 216 may be adjacent the second outer side 204. In various embodiments, ultrasound shots transmitted in the fourth zone 216 shown in FIG. 9 begin nearest the second outer side 204 with each subsequent shot in the fourth zone 216 moving from the second outer side 204 toward the center 206 of the image field 200. In a representative embodiment, the shots transmitted in the fourth zone 216 at step 602 of the second portion of the ultrasound shot sequence 220, 400 may pick up where step 516 of FIG. 12 illustrating the first portion of the ultrasound shot sequence 220, 400 left off.

At step 604, the ultrasound system 100 may determine whether all of the shots of the shot sequence 220, 400 have been fired. For example, the shot sequence 220, 400 may have a first portion and a second portion. The shot sequence 220, 400 may be a pre-defined sequence of ultrasound shots. The first portion of the shot sequence 220, 400 may account for substantially half (defined as 40-60%) of the total shots of the shot sequence 220, 400 and the second portion of the shot sequence 220, 400 may account for the other substantially half of the total shots of the shot sequence 220, 400. If the second portion of the shot sequence 220, 400 has been completed after transmitting the ultrasound shot in the fourth zone 216 of the image field 200 at step 602, ultrasound system 100 at step 604 is finished transmitting ultrasound shots to acquire an ultrasound image frame as defined by step 606. If the ultrasound frame is the last frame to be acquired, the ultrasound system 100 may be finished transmitting ultrasound shots. Alternatively, the ultrasound system 100 may return to FIG. 12 to continue acquiring ultrasound image frames. If the second portion of the shot sequence 220, 400 has not been completed after transmitting the ultrasound shot in the fourth zone 216 of the image field 200 at step 602, ultrasound system 100 at step 604 proceeds to step 608.

At step 608, the probe 104 of the ultrasound system 100 may transmit an ultrasound shot in a first zone 210 of an image field 200. For example, as shown in FIG. 9, the first zone 210 may be adjacent the first outer side 202 on an opposite side of the image field 200 than the third 214 and fourth 216 zones. In various embodiments, ultrasound shots transmitted in the first zone 210 shown in FIG. 9 begin nearest the first outer side 202 with each subsequent shot in the first zone 210 moving from the first outer side 202 toward the center 206 of the image field 200. In a representative embodiment, the shots transmitted in the first zone 210 at step 608 of the second portion of the ultrasound shot sequence 220, 400 may pick up where step 504 of FIG. 12 illustrating the first portion of the ultrasound shot sequence 220, 400 left off. In certain embodiments, the minimum spatial distance from the shot at step 602 to the subsequent shot at step 608 is at least one quarter of the total number of shots in the pre-determined shot sequence 220, 400. For example, if the shot sequence 220, 400 includes a total of 52 shots as illustrated in FIG. 11, the minimum spatial distance from the shot at step 602 to the shot at step 608 is 13 shot positions. In an exemplary embodiment, no dead time is inserted between a reception of echoes from the shot at step 602 and the transmission of the shot at step 608.

At step 610, the ultrasound system 100 may determine whether all of the shots of the shot sequence 220, 400 have been fired. For example, if the second portion of the shot sequence 220, 400 has been completed after transmitting the ultrasound shot in the first zone 210 of the image field 200 at step 608, ultrasound system 100 proceeds to step 610 where the process of transmitting the second portion of the shot sequence 220, 400 to acquire an ultrasound image frame ends. If the ultrasound frame is the last frame to be acquired, the ultrasound system 100 may be finished transmitting ultrasound shots. Alternatively, the ultrasound system 100 may return to FIG. 12 to continue acquiring ultrasound image frames. If the second portion of the shot sequence 220, 400 has not been completed after transmitting the ultrasound shot in the first zone 210 of the image field 200 at step 608, ultrasound system 100 at step 610 proceeds to step 614.

At step 614, the probe 104 of the ultrasound system 100 may transmit an ultrasound shot in a third zone 214 of an image field 200. For example, as shown in FIG. 9, the third zone 214 may be adjacent the center 206 of the image field 200 on an opposite side of the image field 200 than the first 210 and second 212 zones. In various embodiments, ultrasound shots transmitted in the third zone 214 shown in FIG. 9 begin nearest the center 206 of the image field 200 with each subsequent shot in the third zone 214 moving from the center 206 to the second outer side 204, opposite the first outer side 202, of the image field 200. In a representative embodiment, the shots transmitted in the third zone 214 at step 614 of the second portion of the ultrasound shot sequence 220, 400 may pick up where step 510 of FIG. 12 illustrating the first portion of the ultrasound shot sequence 220, 400 left off. In certain embodiments, the minimum spatial distance from the shot at step 608 to the subsequent shot at step 614 is at least one quarter of the total number of shots in the pre-determined shot sequence 220, 400. In an exemplary embodiment, no dead time is inserted between a reception of echoes from the shot at step 608 and the transmission of the shot at step 614.

At step 616, the ultrasound system 100 may determine whether all of the shots of the shot sequence 220, 400 have been fired. For example, if the second portion of the shot sequence 220, 400 has been completed after transmitting the ultrasound shot in the third zone 214 of the image field 200 at step 614, ultrasound system 100 proceeds to step 618 where the process of transmitting the second portion of the shot sequence 220, 400 to acquire an ultrasound image frame ends. If the ultrasound frame is the last frame to be acquired, the ultrasound system 100 may be finished transmitting ultrasound shots. Alternatively, the ultrasound system 100 may return to FIG. 12 to continue acquiring ultrasound image frames. If the second portion of the shot sequence 220, 400 has not been completed after transmitting the ultrasound shot in the third zone 214 of the image field 200 at step 614, ultrasound system 100 at step 616 proceeds to step 620.

At step 620, the probe 104 of the ultrasound system 100 may transmit an ultrasound shot in a second zone 212 of an image field 200. For example, as shown in FIG. 9, the second zone 212 may be adjacent the center 206 of the image field 200 on an opposite side of the image field 200 than the third 214 and fourth 216 zones. In various embodiments, ultrasound shots transmitted in the second zone 212 shown in FIG. 9 begin nearest the center 206 of the image field 200 with each subsequent shot in the second zone 212 moving from the center 206 to the first outer side 202, opposite the second outer side 204, of the image field 200. In a representative embodiment, the shots transmitted in the second zone 212 at step 620 of the second portion of the ultrasound shot sequence 220, 400 may pick up where step 522 of FIG. 12 illustrating the first portion of the ultrasound shot sequence 220, 400 left off. In certain embodiments, the minimum spatial distance from the shot at step 614 to the subsequent shot at step 620 is at least one quarter of the total number of shots in the pre-determined shot sequence 220, 400. In an exemplary embodiment, no dead time is inserted between a reception of echoes from the shot at step 614 and the transmission of the shot at step 620.

At step 622, the ultrasound system 100 may determine whether all of the shots of the shot sequence 220, 400 have been fired. For example, if the second portion of the shot sequence 220, 400 has been completed after transmitting the ultrasound shot in the second zone 212 of the image field 200 at step 620, ultrasound system 100 proceeds to step 624 where the process of transmitting the second portion of the shot sequence 220, 400 to acquire an ultrasound image frame ends. If the ultrasound frame is the last frame to be acquired, the ultrasound system 100 may be finished transmitting ultrasound shots. Alternatively, the ultrasound system 100 may return to FIG. 12 to continue acquiring ultrasound image frames. If the second portion of the shot sequence 220, 400 has not been completed after transmitting the ultrasound shot in the second zone 212 of the image field 200 at step 620, ultrasound system 100 at step 622 returns to step 602 to transmit a next ultrasound shot in the fourth zone 216 of the image field 200. In certain embodiments, the minimum spatial distance from the shot at step 620 to the subsequent shot at step 602 is at least one quarter of the total number of shots in the pre-determined shot sequence 220, 400. In an exemplary embodiment, no dead time is inserted between a reception of echoes from the shot at step 620 and the transmission of the shot at step 602. In various embodiments, the maximum temporal distance between spatially adjacent shot positions is constant, irrespective of the total number of shots in the shot sequence 220, 400. For example, in various embodiments, the maximum temporal distance between spatially adjacent shot positions may be 5. The flow chart 600 continues to repeat steps 602-624 until the second portion of the shot sequence 220, 400 is complete.

Aspects of the present disclosure provide a method 500, 600 and system 100 for providing an improved ultrasound transmit shot sequence 220, 400 to optimize an ultrasound frame rate while minimizing image movement artifacts. In accordance with various embodiments, the method 500, 600 may comprise transmitting 504, 510, 516, 522, 602, 608, 614, 620, by an ultrasound probe 104, a shot sequence 220, 400 having a plurality of shots defining a total number of shots of the shot sequence 220, 400. The shot sequence 220, 400 includes a minimum spatial distance of shot positions from any of the plurality of shots in the shot sequence 220, 400 to a subsequent shot in the shot sequence 220, 400 that is at least a quarter of the total number of shots. The shot sequence 220, 400 includes a maximum temporal distance between spatially adjacent shots of the plurality of shots that is constant irrespective of the total number of shots in the shot sequence 220, 400.

In a representative embodiment, the shot sequence 220, 400 comprises a first portion transmitted for a first substantially half of the total number of shots of the shot sequence 220, 400. The first portion may comprise a first shot pattern that is repeated for the first substantially half of the total number of shots. The first shot pattern may comprise a first sequence of ultrasound shots transmitted in a first zone 210, a third zone 214, a fourth zone 216, and a second zone 212 of an image field 200. The image field 200 may comprise a first outer side 202, a second outer side 204 opposite the first outer side 202, and a center 206 between the first outer side 202 and the second outer side 204. The image field 200 may comprise the first zone 210 adjacent the first outer side 202, the second zone 212 between the first zone 210 and the center 206, the third zone between the center 206 and the fourth zone 216, and the fourth zone 216 adjacent the second outer side 204.

In various embodiments, the shot sequence 220, 400 comprises a second portion transmitted for a second substantially half of the total number of shots of the shot sequence 220, 400. The second portion may comprise a second shot pattern, different from the first shot pattern, that is repeated for the second substantially half of the total number of shots. The second shot pattern may comprise a second sequence of ultrasound shots transmitted in the fourth zone 216, the first zone 210, the third zone 214, and the second zone 212. A starting zone of a first ultrasound shot in the second sequence may depend on an ending zone of a last ultrasound shot in the first sequence.

In an exemplary embodiment, a dead time is not inserted between any two consecutive shots of the plurality of shots transmitted in the shot sequence 220, 400. In a representative embodiment, a first shot 222 in the first zone 210 is transmitted adjacent the first outer side 202 with each subsequent shot in the first zone 210 moving toward the center 206. In various embodiments, a first shot 228 in the second zone 212 is transmitted adjacent the center 206 with each subsequent shot in the second zone 212 moving toward the first outer side 202. In certain embodiments, a first shot 224 in the third zone 214 is transmitted adjacent the center 206 with each subsequent shot in the third zone 214 moving toward the second outer side 204. In an exemplary embodiment, a first shot 226 in the fourth zone 216 is transmitted adjacent the second outer side 204 with each subsequent shot in the fourth zone 216 moving toward the center 206.

Various embodiments provide a system 100 for providing an improved ultrasound transmit shot sequence 220, 400 to optimize an ultrasound frame rate while minimizing image movement artifacts. The system 100 may comprise an ultrasound probe 104 configured to transmit a shot sequence 220, 400 having a plurality of shots defining a total number of shots of the shot sequence 220, 400. The shot sequence 220, 400 comprises a minimum spatial distance of shot positions from any of the plurality of shots in the shot sequence 220, 400 to a subsequent shot in the shot sequence 220, 400 that is at least a quarter of the total number of shots. The shot sequence 220, 400 comprises a maximum temporal distance between spatially adjacent shots of the plurality of shots that is constant irrespective of the total number of shots in the shot sequence 220, 400

In certain embodiments, the shot sequence 220, 400 comprises a first portion transmitted for a first substantially half of the total number of shots of the shot sequence 220, 400. The first portion may comprise a first shot pattern that is repeated for the first substantially half of the total number of shots. The first shot pattern may comprise a first sequence of ultrasound shots transmitted in a first zone 210, a third zone 214, a fourth zone 216, and a second zone 212 of an image field 200. The image field 200 may comprise a first outer side 202, a second outer side 204 opposite the first outer side 202, and a center 206 between the first outer side 202 and the second outer side 204. The image field 200 may comprise the first zone 210 adjacent the first outer side 202, the second zone 212 between the first zone 210 and the center 206, the third zone 214 between the center 206 and the fourth zone 216, and the fourth zone 216 adjacent the second outer side 204.

In a representative embodiment, the shot sequence 220, 400 comprises a second portion transmitted for a second substantially half of the total number of shots of the shot sequence 220, 400. The second portion may comprise a second shot pattern, different from the first shot pattern, that is repeated for the second substantially half of the total number of shots. The second shot pattern may comprise a second sequence of ultrasound shots transmitted in the fourth zone 216, the first zone 210, the third zone 214, and the second zone 212. A starting zone of a first ultrasound shot in the second sequence may depend on an ending zone of a last ultrasound shot in the first sequence.

In various embodiments, a first shot 222 in the first zone 210 is transmitted adjacent the first outer side 202 with each subsequent shot in the first zone 210 moving toward the center 206. In a representative embodiment, a first shot 228 in the second zone 212 is transmitted adjacent the center 206 with each subsequent shot in the second zone 212 moving toward the first outer side 202. In certain embodiments, a first shot 224 in the third zone 214 is transmitted adjacent the center 206 with each subsequent shot in the third zone 214 moving toward the second outer side 204. In an exemplary embodiment, a first shot 226 in the fourth zone 216 is transmitted adjacent the second outer side 204 with each subsequent shot in the fourth zone 216 moving toward the center 206.

Certain embodiments provide a non-transitory computer readable medium having stored thereon, a computer program having at least one code section. The at least one code section is executable by a machine for causing an ultrasound probe 104 to perform steps 500, 600. The steps 500, 600 may comprise transmitting 504, 510, 516, 522, 602, 608, 614, 620, a shot sequence 220, 400 having a plurality of shots defining a total number of shots of the shot sequence 220, 400. The shot sequence 220, 400 comprises a minimum spatial distance of shot positions from any of the plurality of shots in the shot sequence to a subsequent shot in the shot sequence that is at least a quarter of the total number of shots. The shot sequence 220, 400 comprises a maximum temporal distance between spatially adjacent shots of the plurality of shots that is constant irrespective of the total number of shots in the shot sequence 220, 400.

In an exemplary embodiment, the shot sequence 220, 400 comprises a first portion transmitted for a first substantially half of the total number of shots of the shot sequence 220, 400. The first portion may comprise a first shot pattern that is repeated for the first substantially half of the total number of shots. The first shot pattern may comprise a first sequence of ultrasound shots transmitted in a first zone 210, a third zone 214, a fourth zone 216, and a second zone 212 of an image field 200. The image field 200 may comprise a first outer side 202, a second outer side 204 opposite the first outer side 202, and a center 206 between the first outer side 202 and the second outer side 204. The image field 200 may comprise the first zone 210 adjacent the first outer side 202, the second zone 212 between the first zone 210 and the center 206, the third zone 214 between the center 206 and the fourth zone 216, and the fourth zone 216 adjacent the second outer side 204.

In a representative embodiment, the shot sequence 220, 400 comprises a second portion transmitted for a second substantially half of a total number of shots of the shot sequence 220, 400. The second portion may comprise a second shot pattern, different from the first shot pattern, that is repeated for the second substantially half of the total number of shots. The second shot pattern may comprise a second sequence of ultrasound shots transmitted in the fourth zone 216, the first zone 210, the third zone 214, and the second zone 212. A starting zone of a first ultrasound shot in the second sequence may depend on an ending zone of a last ultrasound shot in the first sequence.

In various embodiments, a dead time is not inserted between any two consecutive shots transmitted in the first portion of the shot sequence 220, 400. In certain embodiments, a first shot 222 in the first zone 210 is transmitted adjacent the first outer side 202 with each subsequent shot in the first zone 210 moving toward the center 206. In a representative embodiment, a first shot 228 in the second zone 212 is transmitted adjacent the center 206 with each subsequent shot in the second zone 212 moving toward the first outer side 202. In an exemplary embodiment, a first shot 224 in the third zone 214 is transmitted adjacent the center 206 with each subsequent shot in the third zone 214 moving toward the second outer side 204. In various embodiments, a first shot 226 in the fourth zone 216 is transmitted adjacent the second outer side 204 with each subsequent shot in the fourth zone 216 moving toward the center 206.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for providing an improved ultrasound transmit shot sequence to optimize an ultrasound frame rate while minimizing image movement artifacts.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method comprising:
transmitting, by an ultrasound probe, a shot sequence having a plurality of shots defining a total number of shots of the shot sequence, wherein the shot sequence comprises:
a minimum spatial distance of shot positions from any of the plurality of shots in the shot sequence to a subsequent shot in the shot sequence that is at least a quarter of the total number of shots, and
a maximum temporal distance between spatially adjacent shots of the plurality of shots that is constant irrespective of the total number of shots in the shot sequence.

2. The method of claim 1, wherein:
the shot sequence comprises a first portion transmitted for a first substantially half of the total number of shots of the shot sequence,
the first portion comprises a first shot pattern that is repeated for the first substantially half of the total number of shots,
the first shot pattern comprises a first sequence of ultrasound shots transmitted in a first zone, a third zone, a fourth zone, and a second zone of an image field, and
the image field comprises:
a first outer side,
a second outer side opposite the first outer side,
a center between the first outer side and the second outer side,
the first zone adjacent the first outer side,
the second zone between the first zone and the center,
the third zone between the center and the fourth zone, and
the fourth zone adjacent the second outer side.

3. The method of claim 2, wherein:
the shot sequence comprises a second portion transmitted for a second substantially half of the total number of shots of the shot sequence,
the second portion comprises a second shot pattern, different from the first shot pattern, that is repeated for the second substantially half of the total number of shots,
the second shot pattern comprises a second sequence of ultrasound shots transmitted in the fourth zone, the first zone, the third zone, and the second zone, and
a starting zone of a first ultrasound shot in the second sequence depends on an ending zone of a last ultrasound shot in the first sequence.

4. The method of claim 1, wherein a dead time is not inserted between any two consecutive shots of the plurality of shots transmitted in the shot sequence.

5. The method of claim 2, wherein a first shot in the first zone is transmitted adjacent the first outer side, and wherein each subsequent shot in the first zone moves toward the center.

6. The method of claim 2, wherein a first shot in the second zone is transmitted adjacent the center, and wherein each subsequent shot in the second zone moves toward the first outer side.

7. The method of claim 2, wherein a first shot in the third zone is transmitted adjacent the center, and wherein each subsequent shot in the third zone moves toward the second outer side.

8. The method of claim 2, wherein a first shot in the fourth zone is transmitted adjacent the second outer side, and wherein each subsequent shot in the fourth zone moves toward the center.

9. A system comprising:
an ultrasound probe configured to:
transmit a shot sequence having a plurality of shots defining a total number of shots of the shot sequence, wherein the shot sequence comprises:
a minimum spatial distance of shot positions from any of the plurality of shots in the shot sequence to a subsequent shot in the shot sequence that is at least a quarter of the total number of shots, and
a maximum temporal distance between spatially adjacent shots of the plurality of shots that is constant irrespective of the total number of shots in the shot sequence.

10. The system of claim 9, wherein:
the shot sequence comprises a first portion transmitted for a first substantially half of the total number of shots of the shot sequence,
the first portion comprises a first shot pattern that is repeated for the first substantially half of the total number of shots,
the first shot pattern comprises a first sequence of ultrasound shots transmitted in a first zone, a third zone, a fourth zone, and a second zone of an image field, and the image field comprises:
a first outer side,
a second outer side opposite the first outer side,
a center between the first outer side and the second outer side,
the first zone adjacent the first outer side,
the second zone between the first zone and the center,
the third zone between the center and the fourth zone, and
the fourth zone adjacent the second outer side.

11. The system of claim 10, wherein:
the shot sequence comprises a second portion transmitted for a second substantially half of the total number of shots of the shot sequence,
the second portion comprises a second shot pattern, different from the first shot pattern, that is repeated for the second substantially half of the total number of shots,
the second shot pattern comprises a second sequence of ultrasound shots transmitted in the fourth zone, the first zone, the third zone, and the second zone, and
a starting zone of a first ultrasound shot in the second sequence depends on an ending zone of a last ultrasound shot in the first sequence.

12. The system of claim 10, wherein a first shot in the first zone is transmitted adjacent the first outer side, and wherein each subsequent shot in the first zone moves toward the center.

13. The system of claim 10, wherein a first shot in the second zone is transmitted adjacent the center, and wherein each subsequent shot in the second zone moves toward the first outer side.

14. The system of claim 10, wherein a first shot in the third zone is transmitted adjacent the center, and wherein each subsequent shot in the third zone moves toward the second outer side.

15. The system of claim 10, wherein a first shot in the fourth zone is transmitted adjacent the second outer side, and wherein each subsequent shot in the fourth zone moves toward the center.

16. A non-transitory computer readable medium having stored thereon, a computer program having at least one code section, the at least one code section being executable by a machine for causing an ultrasound probe to perform steps comprising:
transmitting a shot sequence having a plurality of shots defining a total number of shots of the shot sequence, wherein the shot sequence comprises:
a minimum spatial distance of shot positions from any of the plurality of shots in the shot sequence to a subsequent shot in the shot sequence that is at least a quarter of the total number of shots, and
a maximum temporal distance between spatially adjacent shots of the plurality of shots that is constant irrespective of the total number of shots in the shot sequence.

17. The non-transitory computer readable medium of claim 16, wherein:
the shot sequence comprises a first portion transmitted for a first substantially half of the total number of shots of the shot sequence,
the first portion comprises a first shot pattern that is repeated for the first substantially half of the total number of shots,
the first shot pattern comprises a first sequence of ultrasound shots transmitted in a first zone, a third zone, a fourth zone, and a second zone of an image field, and
the image field comprises:
a first outer side,
a second outer side opposite the first outer side,
a center between the first outer side and the second outer side,
the first zone adjacent the first outer side,
the second zone between the first zone and the center,
the third zone between the center and the fourth zone, and
the fourth zone adjacent the second outer side.

18. The non-transitory computer readable medium of claim 17, wherein:
the shot sequence comprises a second portion transmitted for a second substantially half of a total number of shots of the shot sequence,
the second portion comprises a second shot pattern, different from the first shot pattern, that is repeated for the second substantially half of the total number of shots,
the second shot pattern comprises a second sequence of ultrasound shots transmitted in the fourth zone, the first zone, the third zone, and the second zone, and
a starting zone of a first ultrasound shot in the second sequence depends on an ending zone of a last ultrasound shot in the first sequence.

19. The non-transitory computer readable medium of claim 16, wherein a dead time is not inserted between any two consecutive shots transmitted in the first portion of the shot sequence.

20. The non-transitory computer readable medium of claim 16, wherein:
a first shot in the first zone is transmitted adjacent the first outer side, and wherein each subsequent shot in the first zone moves toward the center;
a first shot in the second zone is transmitted adjacent the center, and wherein each subsequent shot in the second zone moves toward the first outer side;
a first shot in the third zone is transmitted adjacent the center, and wherein each subsequent shot in the third zone moves toward the second outer side; and
a first shot in the fourth zone is transmitted adjacent the second outer side, and wherein each subsequent shot in the fourth zone moves toward the center.
